(19) United States
(12) Patent Application Publication
(10) Pub. No.: US 2026/0034047 A1
(43) Pub. Date: Feb. 5, 2026

(54) NATURAL HAIR STYLING COMPOSITION AND METHODS FOR STYLING HAIR

(71) Applicant: L'OREAL, Paris (FR)

(72) Inventors: Rita CHOKSHI, Montclair, NJ (US); Manon CHAUMONTET, Jersey City, NJ (US); Krishna R. RAGHUPATHI, Morganville, NJ (US)

(73) Assignee: L'OREAL, Paris (FR)

(21) Appl. No.: 18/789,490

(22) Filed: Jul. 30, 2024

(51) Int. Cl. A61K 8/9717; A61K 8/04; A61K 8/34; A61K 8/60; A61K 8/73; A61Q 5/06

(52) U.S. Cl. CPC A61K 8/9717; A61K 8/042; A61K 8/345; A61K 8/60; A61K 8/73; A61Q 5/06; A61K 2800/262; A61K 2800/30; A61K 2800/33; A61K 2800/34

(57) ABSTRACT

A hair styling composition is described comprising: (a) carrageenan; (b) glucomannan; (c) pullulan; (d) one or more monosaccharides, disaccharides, sugar alcohols, or combinations thereof; (e) one or more glycols having from 2 to 10 carbon atoms; and (f) water. The composition is preferably a gel having a pH from about 6 to about 8 and is particularly useful in methods for styling hair, including methods for durably shaping hair, improving style, hold, and regularity, imparting shine, and providing frizz control.

### FIELD OF THE DISCLOSURE

The present disclosure pertains to hair styling compositions made with key ingredients that are natural. The hair styling compositions are particularly useful in methods for styling hair, including methods for durably shaping hair, improving style, hold, and regularity, imparting shine, and providing frizz control.

### BACKGROUND

Consumers today are increasingly seeking hair products that not only provide effective styling benefits but are also formulated with natural key ingredients, e.g. thickeners, fixatives, humectants, and the like. There is a growing trend towards formulations that use natural, plant-derived, and sustainable ingredients, driven by concerns about environmental impact and personal health. This shift reflects a broader desire to move away from synthetic and petrochemically-derived ingredients, which are perceived as less environmentally friendly and potentially harmful.

Hair styling products vary widely in form and function, encompassing foams, gels, creams, waxes, mousses, sprays, serums, and aerosols. Each formulation caters to different styling needs and hair types, offering varying levels of benefits depending on the specific composition and application method. However, traditional styling products often suffer from drawbacks such as stickiness upon application and a stiff or “crunchy” feel after drying, which many consumers consider undesirable.

Current advancements in hair product formulations frequently incorporate water-soluble film-forming polymers into hair styling products, many of which are synthetic polymers. These polymers exhibit diverse characteristics depending on their chemical composition; some are inherently water-soluble, while others are modified through processes like neutralization to achieve water solubility. When applied, these polymers form a film on the hair surface that can provide styling hold. However, as the solvent evaporates after application, the concentration of polymers increases, potentially leading to a film that forms residue on the hair and/or flakes therefrom.

The formulation of environmentally-friendly cosmetic products, which are designed and developed considering environmental issues, is becoming a major goal in an effort to meet global challenges. It is therefore essential to propose more sustainable compositions, preparation processes and ingredients to address these environmental concerns. In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

The evolving landscape of hair care products reflects a growing demand for sustainable products that combine effective styling attributes with natural ingredients. These ingredients not only offer functional benefits like styling hold and moisture retention but also appeal to consumers seeking gentler, eco-conscious alternatives. In view of the foregoing, there remains a need for hair styling compositions that harness the benefits of natural ingredients while delivering superior styling performance.

### SUMMARY OF THE DISCLOSURE

The instant disclosure relates to a hair styling compositions, in particular, hair styling compositions that are unique in their ability to impart a variety of desirable properties to hair without reliance on synthetic ingredients. For example, the compositions maintain the shape of hair, achieve long-lasting frizz control, provide strong styling hold, and impart a pleasant texture and body to the hair. Furthermore, hair styled with the compositions has a clean, natural, and light-weight feel. The hair styling composition includes:
(a) carrageenan;
(b) glucomannan;
(c) pullulan;
(d) one or more monosaccharides, disaccharides, sugar alcohols, or combinations thereof;
(e) one or more glycols having from 2 to 10 carbon atoms; and
(f) water.

The hair styling composition is typically a gel with a pH from about 6 to about 8. The components of the hair styling composition are solubilized into a homogenous solution and therefore they are not emulsions or dispersions. If needed, pH adjusters can be used to attain the desired pH. The compositions typically do not include fatty compounds, which are not soluble in water. Rather, the components of composition are soluble in water or can be solubilized in water to form a homogenous solution.

The viscosity of the compositions will vary but is consistent with typical hair gel products. In various embodiments, the hair styling composition has a viscosity from about 5,000 to about 180,000 cps at 25° C. on a Brookfield viscometer using an RV spindle #6 (RVDV2+, spindle 6, 20 rpm). Furthermore, the hair styling compositions are essentially free from synthetic film forming polymers. Furthermore, the hair styling compositions are also preferably essentially free from surfactants, silicones, mono-alcohols having from 1 to 6 carbon atoms, or combinations thereof.

The hair styling composition typically includes one or more monosaccharides, disaccharides, sugar alcohols, or combinations thereof. Nonlimiting examples of monosaccharides includes glucose, fructose, galactose, ribose, deoxyribose, xylose, arabinose, and mannose. Nonlimiting examples of disaccharides include sucrose, lactose, maltose, trehalose, and cellobiose. Nonlimiting examples of sugar alcohols include erythritol, xylitol, sorbitol, mannitol, isomalt, lactitol, maltitol, hydrogenated starch hydrolysates, polyglycitol syrup, arabitol, ribitol, iditol, galactitol, talitol, allitol, dulcitol, glycerol, threitol, isosorbide, sorbitan, xylitan, mannitan, maltitol syrup, glycitol, and sorbitol syrup. Preferred sugar alcohols include isomalt, mannitol, sorbitol, xylitol, lactitol, maltitol, inositol, erythritol, and hydrogenated starch hydrolysates.

Nonlimiting examples of glycols having from 2 to 10 carbon atoms include ethylene glycol, propanediol (propane-1,3-diol), butylene glycol, hexylene glycol, pentylene glycol, propanediol, and diethylene glycol. Propanediol is a particularly preferred glycol.

Additional components may also optionally be included in the hair styling compositions, as set forth throughout the instant disclosure. Likewise, components may be excluded from the hair styling compositions. As already mentioned, the hair styling compositions are preferably essentially free of synthetic film forming polymers, synthetic surfactants, silicones, or combinations thereof.

Finally, hair styling compositions may be used in various methods for styling or treating hair, for example, human hair, including human hair of an individual's head. For example, the compositions particularly useful for: (i) improving or retaining curl definition of hair; (ii) imparting humidity resistance to hair; (iii) reducing hair frizz; (iv) controlling hair volume; (v) styling or shaping hair; (vi) straightening hair; and (vi) improving the appearance of hair.

### DETAILED DESCRIPTION OF THE DISCLOSURE

The hair styling compositions of the instant disclosure includes a unique combination of natural ingredients, for example, carrageenan, glucomannan, and additional natural film forming polymers. The interaction or synergy amongst these components results in compositions that provide desirable softening attributes, holding ability, and styling properties to hair. More specifically, the hair styling compositions contain:
(a) about 0.1 to about 5 wt. % of carrageenan;
(b) about 0.1 to about 5 wt. % of glucomannan;
(c) about 0.1 to about 8 wt. % of pullulan;
(d) about 1 to about 12 wt. % of one or more monosaccharides, disaccharides, sugar alcohols, or combinations thereof;
(e) about 1 to about 12 wt. % of one or more glycols having from 2 to 10 carbon atoms; and
(f) about 70 to about 95 wt. % of water.

The hair treatment composition is preferably a gel, for example, a solubilized, homogenous gel, which means that the composition is not an emulsion or a dispersion but instead, the components of the composition exist in a single, solubilized phase, which is substantially homogenous. The viscosity of the compositions will vary but is consistent with typical hair gel products. In various embodiments, the hair styling composition has a viscosity from about 5,000 to about 180,000 cps at 25° C. on a Brookfield viscometer using an RV spindle #6. In addition, the composition preferably has a pH from about 6 to about 8, which is similar to the pH of healthy hair. Furthermore, in various embodiments, the hair styling compositions is preferably transparent or translucent.

### (a) Carrageenan

Carrageenan is a natural polysaccharide derived from edible seaweeds, primarily from species of red algae belonging to the genera Chondrus, Eucheuma, and Gigartina. It has been used for centuries in various cultures as a thickening and stabilizing agent in foods, pharmaceuticals, and other industrial applications. Carrageenan is extracted through a process that involves boiling the seaweed in water and then filtering and purifying the resulting extract.

Chemically, carrageenan is composed mainly of linear sulfated polysaccharides. The primary building blocks of carrageenan molecules are repeating units of galactose and 3,6-anhydrogalactose (3,6-AG), both of which are linked together by alternating α-(1→3) and β-(1→4) glycosidic bonds. These bonds give carrageenan its unique gel-forming properties, depending on the specific arrangement of these building blocks.

The amount of carrageenan in the hair styling compositions will vary but is typically from about 0.1 to about 5 wt. %, based on the total weight of the hair styling composition. In further embodiments, the hair styling composition includes about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.5 to about 5 wt. %, about 0.5 to about 4 wt. %, about 0.5 to about 3 wt. %, or about 0.5 to about 2 wt. % of carrageenan, based on the total weight of the hair styling composition. Preferably, the hair styling composition includes about 0.2 to about 5 wt. %, more preferably about 0.4 to about 4 wt. %, and more preferably about 0.5 to about 3 wt. % of carrageenan, based on a total weight of the hair styling composition.

### (b) Glucomannan

Glucomannan is a natural polysaccharide derived from the roots of the konjac plant, scientifically known as Amorphophallus konjac or Amorphophallus rivieri. This plant, native to Southeast Asia, particularly Japan, China, and Indonesia, produces tubers rich in glucomannan, which is extracted and purified for various industrial and culinary uses.

Chemically, glucomannan is composed of glucose and mannose units linked together through β-(1→4) glycosidic bonds. The ratio of glucose to mannose units in glucomannan can vary, typically ranging from 1.6:1 to 1.9:1. This polysaccharide is highly water-soluble and forms viscous solutions when mixed with water, which accounts for its unique functional properties.

The amount of glucomannan in the hair styling compositions will vary but is typically from about 0.1 to about 5 wt. %, based on the total weight of the hair styling composition. In further embodiments, the hair styling composition includes about 0.1 to about 4 wt. %, about 0.1 to about 3 wt. %, about 0.1 to about 2 wt. %, about 0.2 to about 5 wt. %, about 0.2 to about 4 wt. %, about 0.2 to about 3 wt. %, about 0.2 to about 2 wt. %, about 0.5 to about 5 wt. %, about 0.5 to about 4 wt. %, about 0.5 to about 3 wt. %, or about 0.5 to about 2 wt. % of glucomannan, based on the total weight of the hair styling composition. Preferably, the hair styling composition includes about 0.2 to about 5 wt. %, more preferably about 0.4 to about 4 wt. %, and more preferably about 0.5 to about 3 wt. % of glucomannan, based on a total weight of the hair styling composition.

### Weight Ratio of (a) to (b)

The amounts of carrageenan and glucomannan and the weight ratio of the carrageenan and the glucomannan relative to one another will vary. For example, in various embodiments, the carrageenan and the glucomannan are in a weight ratio of about 5:1 to about 1:5. In further embodiments, the weight ratio of the carrageenan to the glucomannan is from about 4:1 to about 1:4, about 3:1 to about 1:3, about 2:1 to about 1:2, about 1.5:1 to about 1:1.5, about 1.2:1 to about 1:1.2, or about 1:1. Preferably, the weight ratio of the carrageenan to the glucomannan is from about 3:1 to about 1:3, more preferably from about 2:1 to about 1:2, and even more preferably from about 1.5:1 to about 1:1.5.

### (c) Pullulan

Pullulan is a polysaccharide, a type of carbohydrate, composed of maltotriose units. It is a linear polymer made up of α-D-glucose units linked by α-1,4 and α-1,6 glycosidic bonds and is produced by fermentation of starch by the yeast Aureobasidium pullulans. The inventors found that pullulan provides a variety of benefits to the hair styling compositions, for example, pullulan acts as a film-forming polymer. Films formed are flexible, non-tacky, and long-lasting.

The total amount of pullulan in the hair styling composition can vary but is typically from about 0.1 to about 8 wt. %, based on the total weight of the hair styling composition. In further embodiments, the hair styling composition includes about 0.1 to about 5 wt. %, about 0.1 to about 4 wt. %, about 0.1 to about 3 wt. %, about 0.1 to about 2 wt. %, about 0.2 to about 8 wt. %, about 0.2 to about 5 wt. %, about 0.2 to about 4 wt. %, about 0.2 to about 3 wt. %, about 0.2 to about 2 wt. %, about 0.5 to about 8 wt. %, about 0.5 to about 5 wt. %, about 0.5 to about 4 wt. %, about 0.5 to about 3 wt. %, about 0.5 to about 2 wt. %, about 0.8 to about 8 wt. %, about 0.8 to about 5 wt. %, about 0.8 to about 4 wt. %, about 0.8 to about 3 wt. %, about 0.8 to about 2 wt. %, or about 1 to about 2 wt. % of pullulan, based on the total weight of the hair styling composition. Preferably, the hair styling composition includes about 0.2 to about 5 wt. %, more preferably about 0.4 to about 4 wt. %, and more preferably about 0.5 to about 3 wt. % of pullulan, based on the total weight of the hair styling composition.

### Additional Natural Film-Forming Polymers

The hair styling composition do not require film-forming polymers other than the carrageenan, glucomannan, and pullulan. Nonetheless, one or more additional, natural film-forming polymers may optionally be included or excluded from the hair treatment compositions. Nonlimiting examples of natural film forming polymers include polysaccharide film forming polymers and protein film forming polymers. Nonlimiting examples of polysaccharide film forming polymers include starch, cellulose, alginate, sodium alginate, chitosan, gum Arabic, carrageenan, pullulan, and pectin. Nonlimiting examples of protein film forming polymers include silk fibroin, corn zein, collagen, albumin, keratin, wheat protein, soy protein, and rice protein.

In various embodiment, the hair styling composition preferably includes one or more polysaccharide film forming polymers (other than the carrageenan, glucomannan, and pullulan). Nonlimiting examples of polysaccharide film forming polymers include cellulose, starch, chitosan, chitin, alginate, pectin, gellan gum, xanthan gum, pullulan, bum Arabic, konjac glucomannan, agar, hyaluronic acid, dextran, amylose, and beta-glucan. These polysaccharides vary in their chemical structures but share the common ability to form films through gelation, hydration, or cross-linking mechanisms.

The total amount of the additional natural film-forming polymers, if present, will vary but may be from about 0.01 to about 5 wt. %, based on the total weight of the hair styling composition. In further embodiments, the hair styling composition includes about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.5 to about 5 wt. %, about 0.5 to about 4 wt. %, about 0.5 to about 3 wt. %, or about 0.5 to about 2 wt. % of the additional natural film-forming polymers, based on the total weight of the hair styling composition. Preferably, when the hair styling composition includes one or more additional natural film-forming polymers, the hair styling composition preferably includes about 0.01 to about 5 wt. %, more preferably about 0.05 to about 4 wt. %, and more preferably about 0.1 to about 3 wt. % of the additional natural film-forming polymers, based on the total weight of the hair styling composition.

### (d) Monosaccharides, Disaccharides, and Sugar Alcohols

Monosaccharides, also known as simple sugars, are the most basic units of carbohydrates. Chemically, they are classified as polyhydroxy aldehydes or ketones, characterized by their small size and solubility in water. Structurally, monosaccharides consist of a single sugar unit, typically composed of three to seven carbon atoms.

Nonlimiting examples of useful monosaccharides include glucose, fructose, galactose, mannose, ribose, deoxyribose, xylose, arabinose, lyxose, allose, altrose, talose, gulose, idose, or combinations thereof. Preferred monosaccharides include glucose, fructose, galactose, ribose, mannose, and combinations thereof.

Disaccharides consist of two monosaccharide units joined by a covalent glycosidic bond, which forms between the hydroxyl group (—OH) of one monosaccharide and the anomeric carbon atom (carbon bonded to the oxygen of the carbonyl group) of another monosaccharide. The glycosidic bond can be either a or β depending on the orientation of the linkage.

Nonlimiting examples of disaccharides include sucrose, lactose, maltose, trehalose, cellobiose, gentiobiose, isomaltose, sophorose, laminaribiose, maltulose (isomaltulose), nigerose, turanose, or combinations thereof. Preferred disaccharides include sucrose, lactose, maltose, or combinations thereof.

Sugar alcohols are derived from sugars by replacing the carbonyl group (aldehyde or ketone) with a hydroxyl group (—OH). Thus, sugar alcohols are polyhydroxy alcohols having multiple hydroxyl (—OH) groups attached to their carbon backbone. Nonlimiting examples of sugar alcohols include erythritol, xylitol, sorbitol, mannitol, isomalt, lactitol, maltitol, hydrogenated starch hydrolysates, polyglycitol syrup, arabitol, ribitol, iditol, galactitol, talitol, allitol, dulcitol, glycerol, threitol, isosorbide, sorbitan, xylitan, mannitan, maltitol syrup, glycitol, and sorbitol syrup. Preferred sugar alcohols include erythritol, xylitol, sorbitol, mannitol, isomalt, or combinations thereof.

In a preferred embodiment, the hair styling composition includes one or more sugar alcohols.

The total amount of the one or more monosaccharides, disaccharides, sugar alcohols, or combinations thereof, in the hair styling composition will vary but is typically from about 1 to about 12 wt. %, based on the total weight of the hair styling composition. In further embodiments, the hair styling composition includes from about 1 to about 10 wt. %, about 1 to about 8 wt. %, about 1 to about 6 wt. %, about 2 to about 12 wt. %, about 2 to about 10 wt. %, about 2 to about 8 wt. %, about 3 to about 12 wt. %, about 3 to about 10 wt. %, about 3 to about 8 wt. %, about 4 to about 12 wt. %, about 4 to about 10 wt. %, or about 4 to about 8 wt. % of the one or more monosaccharides, disaccharides, sugar alcohols, or combinations, based on the total weight of the hair styling composition. Preferably, the hair styling composition includes from about 1 to about 12 wt. %, more preferably from about 2 to about 10 wt. %, and even more preferably from about 3 to about 9 wt. % of one or more monosaccharides, disaccharides, sugar alcohols, or combinations, based on the total weight of the hair styling composition.

(e) Glycols Having from 2 to 10 Carbon Atoms

Glycols are a group of chemical compounds that belong to the alcohol family, specifically diols, characterized by having two hydroxyl (—OH) groups attached to different, adjacent carbon atoms within the same molecule. Nonlimiting examples of glycols having from 2 to 10 carbon atoms include ethylene glycol, propylene glycol, propanediol (propane-1,3-diol), butylene glycol, hexylene glycol, pentylene glycol, propanediol, and diethylene glycol. Preferred glycols include propylene glycol, propanediol, butylene glycol, diethylene glycol, and combinations thereof. Propanediol is particularly preferred.

The total amount of the one or more glycols having from 2 to 10 carbon atoms in the hair styling compositions will vary but is typically from about 0.5 to about 12 wt. %, based on the total weight of the hair styling composition. In further embodiments, the hair styling composition includes from about 0.5 to about 10 wt. %, about 0.5 to about 8 wt. %, about 0.5 to about 5 wt. %, about 1 to about 12 wt. %, about 1 to about 10 wt. %, about 1 to about 8 wt. %, about 1 to about 5 wt. %, about 2 to about 12 wt. %, about 2 to about 10 wt. %, about 2 to about 8 wt. %, or about 2 to about 5 wt. % of the one or more glycols having from 2 to 10 carbon atoms, based on the total weigh to the hair styling composition. Preferably, the hair styling composition includes from about 1 to about 12 wt. %, more preferably about 1 to about 8 wt. %, and even more preferably, about 2 to about 6 wt. % of one or more glycols having from 2 to 10 carbon atoms, based on the total weight of the hair styling composition.

### (f) Water

The total amount of water in the hair styling composition will vary but is typically from about 70 to about 95 wt. %, based on the total weight of the hair styling composition. In further embodiments, the hair styling composition includes from about 70 to about 92 wt. %, about 70 to about 90 wt. %, about 70 to about 85 wt. %, about 70 to about 80 wt. %, about 75 to about 95 wt. %, about 75 to about 92 wt. %, about 75 to about 90 wt. %, about 75 to about 85 wt. %, about 80 to about 95 wt. %, about 80 to about 92 wt. %, about 80 to about 90 wt. %, about 85 to about 95 wt. %, about 85 to about 92 wt. %, or about 85 to about 90 wt. % of water, based on the total weight of the hair styling composition. Preferably, the hair styling includes from about 75 to about 95 wt. %, more preferably about 80 to about 95 wt. %, and even more preferably about 85 to about 92 wt. % of water, based on the total weight of the hair styling composition.

### (g) Miscellaneous Ingredients

The hair styling compositions optionally include or excludes (or is essentially free from) one or more miscellaneous ingredients. In a preferred embodiment, the hair styling composition includes one or more miscellaneous ingredients. Miscellaneous ingredients are ingredients that are compatible with the hair styling compositions and do not disrupt or materially affect the basic and novel properties of the compositions. Nonlimiting examples of miscellaneous ingredients include preservatives, fragrances, pH adjusters, salts, chelating agents, amino acids (e.g., taurine), buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, particular materials, etc.) composition colorants, etc. In various embodiments, the miscellaneous ingredients are chosen from preservatives, fragrances, pH adjusters, salts, chelating agents, buffers, composition colorants, and vegetal or botanic extracts.

In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair. Further, for purposes of the instant disclosure, pearlescent agents are considered composition colorants. Pearlescent agents are cosmetic ingredients primarily used to give a shimmering effect or pearlescent effect to the composition. They can be used to enhance the visual appeal of the hair styling composition.

In addition to the above, the components described as optional components throughout the disclosure are can also be considered miscellaneous ingredients if not expressly set forth as an independent component of the hair styling composition. For example, if a particular component is not expressly set forth in a claim that expressly allows for miscellaneous ingredients, such optional components should be considered included in the miscellaneous ingredients unless otherwise specified. Thus, the term “miscellaneous ingredient” is understood as a catch-all phrase representing additional components that may optionally be present in amounts designated for the miscellaneous ingredients.

The amount of the one or more miscellaneous ingredients in the hair styling composition, if present, will vary. Nonetheless, in various embodiments, the hair styling composition includes about 0.01 to about 10 wt. % of the one or more miscellaneous ingredients, based on a total weight of the hair styling composition. In further embodiments, the hair styling composition includes about 0.01 to about 8 wt. %, about 0.01 to about 6 wt. %, about 0.01 to about 5 wt. %, about 0.01 to about 3 wt. %, about 0.05 to about 10 wt. %, about 0.05 to about 8 wt. %, about 0.05 to about 6 wt. %, about 0.05 to about 5 wt. %, about 0.1 to about 10 wt. %, about 0.1 to about 8 wt. %, about 0.1 to about 6 wt. %, about 0.1 to about 5 wt. % of one or more miscellaneous ingredients, based on a total weight of the hair styling composition.

pH

The pH of the hair styling composition will vary but is preferably from about 6 to about 8. Nonetheless, in various embodiments, the hair styling composition has a pH of about 6 to about 7.5, about 6 to about 7, about 6 to about 6.5, about 6.5 to about 8, about 6.5 to about 7.5, or about 6.5 to about 7.

### Viscosity

The viscosity of the hair styling composition can very. Nonetheless, in various embodiments, the viscosity of the hair styling composition is similar to the viscosity of typical hair styling gels known in the art. Preferably, the hair styling gel has a viscosity of about 5,000 to about 180,000 cps at 25° C. on a Brookfield viscometer using an RV spindle #6. In further embodiments, the viscosity of the hair treatment composition is from about 5,000 to about 150,000 cps, about 5,000 to about 120,000 cps, about 5,000 to about 100,000 cps, about 5,000 to about 70,000 cps, about 5,000 to about 50,000 cps, about 10,000 to about 180,000 cps, about 10,000 to about 150,000 cps, about 10,000 to about 120,000 cps, about 10,000 to about 100,000 cps, about 10,000 to about 70,000 cps, about 10,000 to about 50,000 cps, 15,000 to about 180,000 cps, about 15,000 to about 150,000 cps, about 15,000 to about 120,000 cps, about 15,000 to about 100,000 cps, about 15,000 to about 70,000 cps, about 15,000 to about 50,000 cps at 25° C. on a Brookfield viscometer using an RV spindle #6.

### Optional Exclusions

In various embodiments, the hair styling composition is free or essentially free from synthetic polymers.

In various embodiments, the hair styling composition is free or essentially free from anionic surfactants, cationic surfactants, amphoteric surfactants, nonionic surfactants, or combinations thereof.

In various embodiments, the hair styling composition is free from cationic conditioning polymers regardless of whether the cationic conditioning polymers are synthetic or naturally derived (e.g., guar hydroxypropyltrimonium chloride).

In various embodiments, the hair styling compositions are free from silicones, including amino-functionalized silicones.

In various embodiments, the hair styling composition is free or essentially free from waxes. Nonlimiting examples of waxes that may be excluded include beeswax, carnauba wax, candelilla wax, jojoba wax, microcrystalline wax, paraffin wax, ozokerite wax, rice bran wax, sunflower wax, soy wax, shellac wax, Montan wax, lanolin wax, castor wax, bayberry wax, polyethylene wax, polyglyceryl esters, spermaceti wax, Japan wax, orange wax, palm wax, stearyl beeswax, polyethylene glycol (PEG) wax, polyglyceryl-3 beeswax, hydrogenated castor oil, ethylene glycol distearate, olive wax, mimosa wax, Myrica fruit wax, and silica silylate.

In various embodiments, the hair styling compositions are free or essentially free from fatty compounds. Fatty compounds are a diverse group of organic molecules characterized by their insolubility in water (hydrophobic nature) and solubility in nonpolar solvents. Nonlimiting examples of fatty compounds include fatty acids, fatty alcohols, triglycerides, phospholipids, steroids, waxes, oils, glycerides, sphinoglipids, eisosanoids, alkylglycerols, polyketides, fatty acid amides, and ether lipids.

### Methods

Another aspect of the invention pertains to methods for styling hair using the hair styling compositions described herein. The methods generally comprise applying the hair styling compositions described to hair. The hair styling compositions may be useful in a variety of settings, and either for chemically treated or untreated hair. Use on treated hair can include chemically relaxed/straightened hair or chemically dyed or bleached or lightened/highlighted hair. The hair upon which the hair styling composition is applied may bet wet or damp or may be dry. In a preferred embodiment, the hair styling composition is applied to wet or damp hair. In another preferred embodiment, the hair styling composition is applied to dry hair.

The hair styling compositions is typically used as a “leave-on” styling product, i.e., the hair styling composition is applied to the hair and allowed to remain on the hair without rinsing or cleansing the hair after application of the hair styling composition. For example, the hair styling composition may be applied to wet or damp hair and the hair is allowed to dry. The hair can dry naturally or may be dried using drying tools such as a blow dryer. Also, the hair styling composition can be applied to hair (wet, damp, or dry) and the hair can be manipulated into a desired shape or style, for example using fingers, combs, or other styling tools. The hair styling compositions is not rinsed or cleansed from the hair prior to manipulating the hair into the desired shape or style. The hair styling composition is also not rinsed or cleansed from the hair after manipulating the hair into the desired shape or style but instead remains on the shaped/styled hair where it continues to hold/maintain the desired shape/style for an extended period of time (e.g., throughout the day or until the hair is again cleansed according to the individual's regular cleansing and styling routine).

In various embodiments, the hair styling composition is applied to the hair immediately or shortly after cleansing the hair with a shampoo and optionally conditioning the hair with a conditioner.

### EMBODIMENTS

In a preferred embodiment, the hair styling composition comprises, consists essentially of, or consists of:
(a) about 0.1 to about 5 wt. %, preferably about 0.2 to about 4 wt. %, and more preferably, about 0.5 to about 3 wt. % of carrageenan;
(b) about 0.1 to about 5 wt. %, preferably about 0.2 to about 4 wt. %, and more preferably, about 0.5 to about 3 wt. % of glucomannan;
   wherein (a) and (b) are preferably in a weight ratio of about 4:1 to about 1:4, more preferably about 3:1 to about 1:3, and even more preferably about 2:1 to about 1:2;
(c) about 0.1 to about 8 wt. %, preferably about 0.5 to about 5 wt. %, and more preferably about 0.5 to about 3 wt. % of pullulan;
(d) about 1 to about 12 wt. %, preferably about 2 to about 10 wt. %, and more preferably about 3 to about 8 wt. % of one or more monosaccharides, disaccharides, sugar alcohols, or combinations thereof, wherein preferably:
   the one or more monosaccharides are selected from glucose, fructose, galactose, ribose, and deoxyribose;
   the one or more disaccharides are selected from sucrose, lactose, maltose, trehalose, and cellobiose; and
   the one or more sugar alcohols are selected from maltitol, sorbitol, ribitol, mannitol, arabitol, galactitol, xylitol, erythritol, isomalt, lactitol, and hydrogenated starch hydrolysates;
(e) about 1 to about 12 wt. %, preferably about 2 to about 10 wt. %, more preferably about 1 to about 8 wt. %, and even more preferably about 2 to about 6 wt. % of one or more glycols having from 2 to 10 carbon atoms, wherein the one or more glycols are preferably selected from ethylene glycol, propylene glycol, propanediol (propane-1,3-diol) butylene glycol, hexylene glycol, caprylyl glycol, pentylene glycol, propanediol, and diethylene glycol, preferably wherein the one or more glycols comprises or consists of propanediol;
(f) about 70 to about 95 wt. %, preferably about 80 to about 95 wt. %, more preferably about 80 to about 92 wt. % of water; and
(g) optionally, about 0.01 to about 10 wt. %, preferably about 0.1 to about 8 wt. %, more preferably about 1 to about 5 wt. % of one or more miscellaneous ingredients, wherein the one or more miscellaneous ingredients are preferably selected from preservatives, fragrances, chelating agents, antioxidants, flavonoids, vitamins, botanical extracts, UV filtering agents, proteins, protein hydrolysates, protein isolates, fillers, and composition colorants;
   wherein the composition is a gel,
   the composition has a pH of about 6 to about 8, and
   all weight percentages are based on a total weight of the hair styling composition.

In a further preferred embodiment, the hair styling composition comprises, consists essentially of, or consists of:
(a) about 0.1 to about 5 wt. %, preferably about 0.2 to about 4 wt. %, and more preferably, about 0.5 to about 3 wt. % of carrageenan;
(b) about 0.1 to about 5 wt. %, preferably about 0.2 to about 4 wt. %, and more preferably, about 0.5 to about 3 wt. % of glucomannan;
   wherein (a) and (b) are preferably in a weight ratio of about 4:1 to about 1:4, more preferably about 3:1 to about 1:3, and even more preferably about 2:1 to about 1:2;
(c) about 0.1 to about 8 wt. %, preferably about 0.5 to about 5 wt. %, and more preferably about 0.5 to about 3 wt. % of pullulan;
(d) about 1 to about 12 wt. %, preferably about 2 to about 10 wt. %, and more preferably about 3 to about 8 wt. % of one or more sugar alcohols, wherein preferably, the one or more sugar alcohols are selected from maltitol, sorbitol, ribitol, mannitol, arabitol, galactitol, xylitol, erythritol, isomalt, lactitol, or hydrogenated starch hydrolysates, preferably wherein the one or more sugar alcohols comprises or consists of sorbitol;
(e) about 1 to about 12 wt. %, preferably about 2 to about 10 wt. %, more preferably about 1 to about 8 wt. %, and even more preferably about 2 to about 6 wt. % of one or more glycols having from 2 to 10 carbon atoms, wherein the one or more glycols are preferably selected from ethylene glycol, propylene glycol, propanediol (propane-1,3-diol), butylene glycol, hexylene glycol, pentylene glycol, propanediol, and diethylene glycol, more preferably wherein one or more glycols comprise or consist of propanediol;
(f) about 70 to about 95 wt. %, preferably about 80 to about 95 wt. %, more preferably about 80 to about 92 wt. % of water; and
(g) optionally, about 0.01 to about 10 wt. %, preferably about 0.1 to about 8 wt. %, more preferably about 1 to about 5 wt. % of one or more miscellaneous ingredients, wherein the one or more miscellaneous ingredients are preferably selected from preservatives, fragrances, chelating agents, antioxidants, flavonoids, vitamins, botanical extracts, UV filtering agents, proteins, protein hydrolysates, protein isolates, fillers, or composition colorants;
   wherein the composition is a gel,
   the composition has a pH of about 6 to about 8, and
   all weight percentages are based on a total weight of the hair styling composition.

In a further preferred embodiment, the hair styling composition comprises, consists essentially of, or consists of:
(a) about 0.1 to about 5 wt. %, preferably about 0.2 to about 4 wt. %, and more preferably, about 0.5 to about 3 wt. % of carrageenan;
(b) about 0.1 to about 5 wt. %, preferably about 0.2 to about 4 wt. %, and more preferably, about 0.5 to about 3 wt. % of glucomannan;
   wherein (a) and (b) are preferably in a weight ratio of about 4:1 to about 1:4, more preferably about 3:1 to about 1:3, and even more preferably about 2:1 to about 1:2;
(c) about 0.1 to about 8 wt. %, preferably about 0.5 to about 5 wt. %, and more preferably about 0.5 to about 3 wt. % of pullulan;
(d) about 1 to about 12 wt. %, preferably about 2 to about 10 wt. %, and more preferably about 3 to about 8 wt. % of one or more sugar alcohols selected from maltitol, sorbitol, ribitol, mannitol, arabitol, galactitol, xylitol, erythritol, isomalt, lactitol, or hydrogenated starch hydrolysates, preferably wherein the one or more sugar alcohols comprises or consists of sorbitol;
(e) about 1 to about 12 wt. %, preferably about 2 to about 10 wt. %, more preferably about 1 to about 8 wt. % of propanediol (propane-1,3-diol);
(f) about 70 to about 95 wt. %, preferably about 80 to about 95 wt. %, more preferably about 80 to about 92 wt. % of water; and
(g) optionally, about 0.01 to about 10 wt. %, preferably about 0.1 to about 8 wt. %, more preferably about 1 to about 5 wt. % of one or more miscellaneous ingredients, wherein the one or more miscellaneous ingredients are preferably selected from preservatives, fragrances, chelating agents, antioxidants, flavonoids, vitamins, botanical extracts, UV filtering agents, proteins, protein hydrolysates, protein isolates, fillers, and composition colorants;
   wherein the composition is a gel,
   the composition has a pH of about 6 to about 8, and
   all weight percentages are based on a total weight of the hair styling composition.

The hair styling compositions set forth above are preferably free or essentially free from synthetic polymers.

The hair styling compositions set forth above are preferably free or essentially free from anionic surfactants, cationic surfactants, amphoteric surfactants, nonionic surfactants, or combinations thereof.

The hair styling compositions set forth above are preferably free from cationic conditioning polymers regardless of whether the cationic conditioning polymers are synthetic or naturally derived (e.g., guar hydroxypropyltrimonium chloride).

The hair styling compositions set forth above are preferably free from silicones, including amino-functionalized silicones.

The hair styling compositions set forth above are preferably free or essentially free from waxes. Nonlimiting examples of waxes that may be excluded include beeswax, carnauba wax, candelilla wax, jojoba wax, microcrystalline wax, paraffin wax, ozokerite wax, rice bran wax, sunflower wax, soy wax, shellac wax, Montan wax, lanolin wax, castor wax, bayberry wax, polyethylene wax, polyglyceryl esters, spermaceti wax, Japan wax, orange wax, palm wax, stearyl beeswax, polyethylene glycol (PEG) wax, polyglyceryl-3 beeswax, hydrogenated castor oil, ethylene glycol distearate, olive wax, mimosa wax, Myrica fruit wax, and silica silylate.

The hair styling compositions set forth above are preferably free or essentially free from fatty compounds. Fatty compounds are a diverse group of organic molecules characterized by their insolubility in water (hydrophobic nature) and solubility in nonpolar solvents. Nonlimiting examples of fatty compounds include fatty acids, fatty alcohols, triglycerides, phospholipids, steroids, waxes, oils, glycerides, sphinoglipids, eisosanoids, alkylglycerols, polyketides, fatty acid amides, ether lipids, or combinations thereof.

### EXAMPLES

Various changes can be made in the above-compositions and methods without departing from the scope of the invention. Accordingly, it is intended that the entire disclosure in the above description and in the examples given below, is illustrative and not limiting.

### Example 1

### Inventive Hair Styling Compositions

| | | | A | B | C | D | E |
|---|---|---|---|---|---|---|---|
| (a) | Carrageenan | CARRAGEENAN | 0.75 | 0.75 | 0.75 | 0.75 | 0.75 |
| (b) | Glucomannan | GLUCOMANNAN | 0.75 | 0.75 | 0.75 | 0.75 | 0.75 |
| (c) | Natural Film | PULLULAN | 1 | 1 | 0.5 | 0.5 | 1 |
| | Forming Polymer | ACACIA SENEGAL GUM | | | | | 0.01 |
| (d) | Sugar Alcohol | SORBITOL | 4 | 7.5 | 4 | 7.5 | 7.5 |
| (e) | Glycol | PROPANEDIOL | 2.5 | 2.5 | 2.5 | 2.5 | 2.5 |
| | | (PROPANE-1,3-DIOL) | | | | | |
| (g) | Miscellaneous | PRESERVATIVE, | ≤5 | ≤5 | ≤5 | ≤5 | ≤5 |
| | | FRAGRANCE, pH | | | | | |
| | | ADJUSTERS, ETC. | | | | | |
| (f) | Water | WATER | 90 | 86.5 | 90.5 | 87 | 86.5 |

| | |
|---|---|
| pH | ~6-8 |

The compositions were prepared by first premixing the carrageenan, glucomannan, and pullulan, and propanediol. Sorbitol was dissolved in the water, and the premixture of the carrageenan, glucomannan, pullulan, and propanediol was combined with the water/sorbitol solution. The additional ingredients were added, and the composition mixed to afford a solubilized, homogenous gel.

Polysaccharides other than carrageenan were investigated but they failed to provide satisfactory results. For example, alginate was used instead of carrageenan in a 1:1 ratio with glucomannan. The alginate, however, had a negative impact on pH. Xanthan gum was used instead of carrageenan in a 1:1 ratio with glucomannan. The xanthan gum, however, failed to provide sufficient viscosity. Therefore, the amount of xanthan gum was increased to a ratio of 4:1 (xanthan gum: glucomannan) and a clumpy gel was formed that lacked transparency. Pectin was used instead of carrageenan in a 1:1 ratio with glucomannan. The pectin, however, was difficult to disperse.

### Example 2

### Comparative Testing

Inventive Composition B from Example 1 was compared with a popular Commercial Benchmark Product known for its desirable styling qualities. The Commercial Benchmark Product lists the following ingredients:
Aqua, Propylene Glycol, Sorbitol, Ammonium Polyacryldimethyltauramide, VP/Acrylates/Lauryl Methacrylate Copolymer, Peg-40 Hydrogenated Castor Oil, Magnesium Gluconate, Triethanolamine, Chlorphenesin, Calcium Gluconate, Limonene, Pentasodium Pentetate, Benzyl Salicylate, Benzyl Alcohol, Linalool, Methylparaben, Butylphenyl Methylpropional, Perfume.

The Commercial Benchmark Product, unlike Inventive Composition B, includes synthetic polymers.

A volunteer's hair was cleansed with a standard shampoo and towel dried. Half of the volunteer's head of hair was treated with the Commercial Benchmark Product and the other half of the volunteer's head of hair was treated with Inventive Composition B. Equal amounts of both compositions (amounts of about 2-3 grams) were applied to the hair, uniformly spread throughout the hair, and the hair was styled with the fingers. An expert evaluator assessed the hair for a variety of cosmetic attributes immediately after styling, 3-4 hours after styling, and after washing the compositions from the hair. Hair treated with Inventive Composition B was compared with the hair treated with the Commercial Benchmark Product as follows:
<Indicates that Inventive Composition B did not perform as well as the Commercial Benchmark Product
=Indicates that Inventive Composition B performed on par with the Commercial Benchmark Product
>Indicates that that Inventive Composition B performed better than the Commercial Benchmark Product

The results are shown in the table below.

Immediately After Styling

| | |
|---|---|
| Distribution | = |
| Ease of Passing Fingers | = |
| Ease of Styling/Shaping | > |
| Stickiness | < |
| Ease of Removal from Hands | > |
| Shine | = |
| Wet Appearance | = |
| Thick Wicks/Clumping | > |
| Firmness | = |
| Coating | = |

After 3-4 Hour

| | |
|---|---|
| Shine | = |
| Wet Effect | = |
| Thick Wicks/Clumping | = |
| Firmness | > |
| Styling Hold | > |
| Shape | = |
| Residue | < |

After Cleansing

| | |
|---|---|
| Clean Feel After Washing | > |

The results show that despite being formulated with natural ingredients, Inventive Composition B performed in parity or better than the Commercial Benchmark Product in nearly all attributed tested. For instance, it was easier to style the hair with Inventive Composition B. Also, Inventive Composition was more easily rinsed from the hands. Hair treated with Inventive Composition B suffered fewer thick wicks and less clumping than hair treated with the Comparative Benchmark Product. “Thick wicks or clumping” refers to chunks of hair that have become bound together, which is undesirable.

After 3-5 hours, hair treated with Inventive Composition B was firmer and showed better styling hold. Finally, after washing the compositions from the hair, hair treated with Inventive Composition B exhibited a “cleaner” feel.

It was surprising to find that Inventive Composition B, containing natural ingredients, performed in parity or better than the Commercial Benchmark Product containing synthetic polymers, which are known for their excellent styling attributes. It is known that natural ingredients do not typically perform as well as synthetic ingredients in hair styling compositions. This is because synthetic ingredients are engineered to ensure they provide desired attributes. The number of different synthetic ingredients is boundless. In contrast, the number of natural ingredients is finite. If natural ingredients are chemically modified to attain a desired property, the modified ingredient is no longer natural.

The foregoing disclosure illustrates and describes embodiments of the invention. The disclosure shows and describes only the preferred embodiments, but it is understood that the invention is useable in various other combinations, modifications, and environments and is capable of changes or modifications within the scope of the inventive concepts expressed herein, commensurate with the above teachings and/or the skill or knowledge of the relevant art. The embodiments described above are further intended to explain best modes known by applicant and to enable others skilled in the art to utilize the disclosure. This description is not intended to limit the invention.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” or “a combination thereof” also relates to “mixtures thereof” or “combinations thereof.” Throughout the disclosure, the term “a combination thereof” may be used following a list of elements as shown in the following example where letters A-F represent the elements: “one or more elements selected from A, B, C, D, E, F, or combination thereof.” The term, “combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a combination thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The surfactants referred to throughout the disclosure may include those having a counter-ion such as an alkali metal, alkaline earth metal, or ammonium counterion. This list of counterions, however, is non-limiting. Regardless of whether the possibility of a salt for a particular component is mentioned, both the salt and the non-salt form of the component are intended, unless stated otherwise.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions can be modified in all instances by the term “about,” meaning within +/−10% of the indicated number. For example, an amount of “about 1 wt.” can include an amount as low as 0.90 wt. % or as high as 1.1. Similarly, an amount of “about 50” can include an amount as low as 45 wt. % and as high as 55 wt. %.

Some of the various categories of ingredients identified for the hair styling composition may overlap. In such cases where overlap may exist and the hair styling composition includes two overlapping ingredients (or more than two overlapping ingredients), an overlapping ingredient does not represent more than one claimed component. As an example, menthol is known in the art as both an “cooling agent” and a “terpene.” If a claimed composition referenced both a cooling agent and a terpene, menthol can serve as only the cooling agent or as only the terpene. Menthol cannot simultaneously serve as both the cooling agent and the terpene.

All percentages, parts, and ratios herein are based upon the total weight of the compositions, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

A “rinse-off” product refers to a composition that is rinsed and/or washed from the hair with water either after or during the application of the composition onto the hair, and before drying and/or styling the hair. At least a portion of the composition is removed from the hair during the rinsing and/or washing.

A “leave-on” product refers to a composition that is not rinsed and/or washed from the hair after or during application of the composition onto the hair. The composition remains on the hair during drying and/or throughout styling.

All components positively set forth in the instant disclosure can be negatively excluded from the claims and from the compositions and methods described throughout the disclosure In other words, the compositions of the instant disclosure may be free or essentially free of any one or more of the components positively set forth in the instant disclosure for possible inclusion into the compositions.

The term “substantially free” or “essentially free” as used herein means the specific material may be present in small amounts that do not materially affect the basic and novel characteristics of the claimed invention. For instance, there may be less than 1% by weight of a specific material added to a composition, based on the total weight of the compositions (provided that an amount of 1% or less by weight does not materially affect the basic and novel characteristics of the claimed invention). Similarly, when a composition is essentially free from a particular element, the composition may include 1 wt. % or less, 0.5 wt. % or less, 0.1 wt. % or less, 0.05 wt. % or less, 0.01 wt. % or less, or none of the specified material. As an, if a composition is “substantially free” or “essentially free” from ethanol, the composition may optionally include ethanol in an amount up to 1 wt. %, based on the total weight of the composition, provided an amount of up to 1 wt. % does not materially affect the basic and novel characteristics of the composition. Furthermore, the composition may include 0.5 wt. % or less, 0.1 wt. % or less, 0.05 wt. % or less, 0.01 wt. % or less, or no ethanol.

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition

The term “translucent” with respect to a translucent composition indicates that the composition has a transmittance of at least 50% at a wavelength of 600 nm, for example measured using a Lambda 40 UV-visible spectrometer. A human cannot likely see through a translucent composition, for example, and read the text on the other side of a clear glass or clear plastic bottle containing the composition. Rather, the text is usually blurred and difficult or not possible to read, yet movement and structure can normally be identified.

All publications and patent applications cited throughout the disclosure are incorporated herein by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair styling composition comprising:
(a) about 0.1 to about 5 wt. % of carrageenan;
(b) about 0.1 to about 5 wt. % of glucomannan;
(c) about 0.1 to about 8 wt. % pullulan;
(d) about 1 to about 12 wt. % of one or more monosaccharides, disaccharides, sugar alcohols, or combinations thereof;
(e) about 1 to about 12 wt. % of one or more glycols having from 2 to 10 carbon atoms; and
(f) about 70 to about 95 wt. % of water;
wherein the composition is a gel,
the composition has a pH of about 6 to about 8, and
all weight percentages are based on a total weight of the hair styling composition.

2. The hair styling composition of claim 1, wherein (a) and (b) are in a weight ratio of about 4:1 to 1:4.

3. The hair styling composition of claim 1, wherein:
the one or more monosaccharides are selected from glucose, fructose, galactose, ribose, and deoxyribose;
the one or more disaccharides are selected from sucrose, lactose, maltose, trehalose, and cellobiose; and
the one or more sugar alcohols are selected from maltitol, sorbitol, ribitol, mannitol, arabitol, galactitol, xylitol, erythritol, isomalt, lactitol, and hydrogenated starch hydrolysates.

4. The hair styling composition of claim 3 comprising the one or more sugar alcohols.

5. The hair styling composition of claim 4 comprising the one or more sugar alcohols selected from maltitol, sorbitol, ribitol, mannitol, arabitol, galactitol, xylitol, erythritol, isomalt, lactitol, and hydrogenated starch hydrolysates.

6. The hair styling composition of claim 1, wherein one or more monosaccharides, disaccharides, sugar alcohols, or combinations thereof comprises or consists of sorbitol.

7. The hair styling composition of claim 1, wherein the one or more glycols having from 2 to 10 carbon atoms are selected from ethylene glycol, propylene glycol, propanediol (propane-1,3-diol), butylene glycol, hexylene glycol, pentylene glycol, propanediol, and diethylene glycol.

8. The hair styling composition of claim 7, wherein the one or more glycols having from 2 to 10 carbon atoms comprises or consists of propanediol.

9. The hair styling composition of claim 1, wherein the hair styling composition is free from synthetic polymers.

10. The hair styling composition of claim 1, wherein the composition is free from surfactants.

11. The hair styling composition of claim 1, wherein the composition is free from silicones.

12. The hair styling composition of claim 1, wherein the composition is free from mono-alcohols having from 1 to 6 carbon atoms.

13. The hair styling composition of claim 1 having a viscosity of from about 5,000 to about 60,000 cps at 25° C. on a Brookfield viscometer using an RV spindle #6.

14. The hair styling composition of claim 1, wherein the hair styling composition is transparent or translucent.

15. The hair styling composition of claim 1, wherein the composition is solubilized, homogenous gel.

16. A hair styling composition comprising:
(a) about 0.1 to about 2 wt. % of carrageenan;
(b) about 0.1 to about 2 wt. % of glucomannan;
wherein (a) and (b) are in a weight ratio of about 2:1 to about 1:2;
(c) about 1 to about 5 wt. % of pullulan;
(d) about 2 to about 10 wt. % of sorbitol;
(e) about 1 to about 5 wt. % of one or more glycols selected from propylene glycol, propanediol, butylene glycol, pentylene glycol, hexylene glycol, dipropylene glycol, diethylene glycol, and diglycerol;
(f) optionally, up to 5 wt. % of one or more miscellaneous ingredients;
wherein the composition is a gel,
the composition has a pH of about 6 to about 8, and
all weight percentages are based on a total weight of the hair styling composition.

17. The hair styling composition of claim 16, wherein the hair styling composition is free from synthetic polymers and free from silicones.

18. The hair styling composition of claim 16, wherein the one or more glycols comprises or consists of propanediol.

19. The hair styling composition of claim 16, wherein (a) and (b) are in a weight ratio of about 3:1 to about 1:3.

20. A method for styling hair comprising applying the hair styling composition of claim 1 to the hair.
